(19) United States
(12) Patent Application Publication
(10) Pub. No.: US 2026/0174643 A1
(43) Pub. Date: Jun. 25, 2026

(54) LIQUID CONTAMINANT ABSORPTION DEVICE AND METHODS OF USE

(71) Applicant: SETATECH USA, INC., Homestead, FL (US)

(72) Inventor: Blair BLACKER, Homestead, FL (US)

(21) Appl. No.: 19/125,937

(22) PCT Filed: Jul. 23, 2024
(86) PCT No.: PCT/US2024/039088

§ 371 Date: Apr. 30, 2025

Related U.S. Application Data
(60) Provisional application No. 63/528,442, filed on Jul. 24, 2023.

(51) Int. Cl. A61K 8/23; A61K 8/19; A61K 8/46; A61K 8/49; A61K 8/898

(52) U.S. Cl. CPC A61K 8/23; A61K 8/19; A61K 8/463; A61K 8/4926; A61K 8/898

(57) ABSTRACT

Devices and methods are provided that are suited for containing and absorbing liquid contaminants in or on aqueous liquids. The device includes mammalian hair and at least one scrim. The mammalian hair may be disposed on or wrapped around the scrim. The scrim may include at least one connection means at each of a first end portion and second end portion of the scrim.

### BACKGROUND OF THE DISCLOSURE

Oil and other liquids having a specific gravity which is less than water are frequently transported by boats and ships. Oil and other potentially hazardous liquids are transported across or through water by pipelines or similar conduits. Such liquid materials are loaded onto ships by means of pipelines, often requiring a connection of a pipeline to the ship.

Spills of oil and other liquid contaminant which float on water are common. Shipwrecks or hazards at sea or in waterways can cause oil or other materials to be spilled or otherwise accidentally discharged into waterways. Even vessels which are carrying oil as cargo are powered by diesel fuels or other petroleum fuels, and fuels and engine lubricants may be accidentally discharged into water. Further, pipelines and other conduits which cross waterways are subject to breakage, subjecting the waterway to pollution from discharge of the oil or other material into a waterway. Frequently, oil or other liquid materials are loaded onto ships as cargo by means of pipelines and similar conduits. Cargo ships and pipelines may be subject to damage causing oil spillage in waterways. There remains a need for devices and methods to address such issues.

### SUMMARY OF THE DISCLOSURE

The present disclosure is directed to devices and methods for containing and absorbing liquid contaminants in or on aqueous liquids. The devices and methods provided herein are particularly suited for containing and absorbing oil on an aqueous surface.

According to one embodiment, a device for containing and one or more absorbing liquid contaminant in or on one or more aqueous liquid is provided that includes mammalian hair and at least one flotation device encased within a mammalian hair mat. The flotation device includes a first end portion and a second end portion. The flotation device includes at least one connection means such as ringlets or grommets at each of the first end portion and second end portion.

According to another embodiment, a device for containing and absorbing liquid contaminant in or on an aqueous liquid is provided that includes mammalian hair and at least one flotation device that encases mammalian hair. The mammalian hair may be present within or encased by the flotation device. The mammalian hair may be present as a mammalian hair mat. The flotation device includes a first end portion and a second end portion. The flotation device includes at least one connection means such as ringlets or grommets at each of the first end portion and second end portion.

According to another embodiment, a device for containing and absorbing liquid one or more contaminant in or on one or more aqueous liquid is provided that includes mammalian hair and a scrim. The mammalian hair is applied to two sides or otherwise wrapped around the scrim. The scrim is exposed (i.e., not covered or wrapped in hair) at a first end portion and second end portion. Alternatively, the scrim may be exposed on all ends or edges. The scrim may include one or more connection means such as ringlets or grommets at each of the first end portion and second end portion. According to one embodiment, the scrim may include at least one connection means at a corner portion of the scrim (e.g., connection means at all four corners).

A method for containing one or more liquid contaminants in or on one or more aqueous liquids is provided. The method may include the step of deploying a device that includes mammalian hair and at least one scrim, the scrim having a first end portion and a second end portion. The mammalian hair is disposed on or wrapped around the scrim. The scrim includes at least one connection means at each of the first end portion and second end portion. The device is particularly suited to contain one or more liquid contaminants in or on one or more aqueous liquids. According to one embodiment, the device absorbs the one or more liquid contaminants. According to one embodiment, the mammalian hair is utilized as a mammalian hair mat. According to one embodiment, the mammalian hair mat exhibits a hair density of from about 0.02 to about 3 grams of mammalian hair per cubic inch. According to one embodiment, the mammalian hair mat is capable of or otherwise suit for holding and retaining from generally about 0.02 to generally about 15 grams of liquid contaminant per cubic inch of mammalian hair. According to one embodiment, the mammalian hair mat further includes at least one natural fiber from the group including human hair, animal hair, coconut fiber, seaweed, kelp, cotton, corn silk and peat moss. According to one embodiment, the mammalian hair mat may include a plurality of man-made fibers.

The summary of the disclosure is provided as a general introduction to some of the embodiments of the disclosure and is not intended to be limiting. Additional example embodiments including variations and alternative configurations of the disclosure are provided herein.

### BRIEF DESCRIPTION OF THE DRAWINGS

FIG. 1 illustrates a device for containing and absorbing liquid contaminant in or on an aqueous liquid according to one embodiment.

FIG. 2A illustrates a side view of an alternative device for containing and absorbing liquid contaminant in or on an aqueous liquid according to one embodiment.

FIG. 2B illustrates a top view of the alternative device of FIG. 2A.

FIG. 3 illustrates a side view of an alternative device for containing and absorbing liquid contaminant in or on an aqueous liquid according to one embodiment.

### DETAILED DESCRIPTION OF THE DISCLOSURE

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

As used herein the term “contaminant” refers to a polluting substance present in an aqueous environment.

The use of “a” or “an” are employed to describe elements and components described herein. This is done merely for convenience and to give a general sense of the scope of the disclosure. This description should be read to include one or at least one and the singular also includes the plural unless it is obvious that it is meant otherwise.

### Devices Use/Overview

Various devices for containing and absorbing liquid contaminant in or on an aqueous liquid is provided. The devices provided herein may be used on any suitable aqueous surface where a liquid contaminant is present in or on an aqueous surface. Such an aqueous surface may be located on a freshwater body of water or saltwater body of water. Exemplary bodies of water include rivers, lakes, ponds, seas and oceans. The devices provided herein may be deployed after a liquid contaminant is found present in or on an aqueous liquid (such as after an oil spill). According to one embodiment, the liquid contaminant is less dense than the aqueous liquid. According to one embodiment, the liquid contaminant comprises oil. The oil includes crude oil such as typically transported via pipeline or on tanker ships.

### Mammalian Hair

According to one embodiment, the devices provided herein may utilize mammalian hair (such as in the form of a mammalian hair mat). The mammalian hair may be obtained from human or other suitable mammal. Human hair may be obtained from barbers or salons that are disposing hair that may be otherwise sent to decompose in a land fill.

According to one embodiment, the mammalian hair of the devices provided herein may be utilized as a mammalian hair mat that includes a plurality of individual mammalian hairs intertwined. According to one embodiment, the mammalian hair mat exhibits a hair density of from about 0.02 grams to about 3 grams of mammalian hair per cubic inch. According to one embodiment, the mammalian hair mat exhibits a hair density of from about 1.0 pound of mammalian hair per cubic foot to about 2.00 pounds of mammalian hair per cubic foot.

According to one embodiment, the mammalian hair mat absorbs at least about one (1) gallon of liquid contaminant per five (5) pounds of mammalian hair. According to one embodiment, the mammalian hair mat is capable of holding and retaining from generally about 0.02 grams of liquid contaminant per cubic inch of mammalian hair to about 15 grams of liquid contaminant per cubic inch of mammalian hair.

According to one embodiment, the mammalian hair mat further includes at least one natural fiber from the group including human hair, animal hair, coconut fiber, seaweed, kelp, cotton, corn silk and peat moss. According to one embodiment, the mammalian hair mat includes a plurality of man-made fibers.

### Flotation Device

According to one embodiment, the devices provided herein may include a flotation device that, in turn, includes at least one closed-cell foam. According to one embodiment, the flotation device includes polyethylene foam, urethane foam, polyurethane foam, or a combination thereof. According to one embodiment, the flotation device is substantially cylindrical in shape. According to one embodiment, the flotation device is mold and mildew resistant. According to one embodiment, the mammalian hair mat is secured to the flotation device. According to one embodiment, the mammalian hair mat is secured to the flotation device via one or more zip ties or other suitable security means.

According to one embodiment, the flotation device includes a first end portion and a second end portion. The flotation device may include at least one connection means at each of the first end portion and second end portion. According to one embodiment, the at least one connection means includes a ringlet, the ringlet adapted to receive an attachment means. According to one embodiment, the at least one connection means at each of the first end portion and second end portion includes an eye hook that is adapted to receive an attachment means.

According to one embodiment, the attachment means includes a rope or other similar device that is suitable to attaching one or more devices as provided herein together or for controlling the placement and movement of the devices.

### Scrim

According to one embodiment, the devices provided herein may utilize at least one scrim. The scrim may be formed from a strong fabric (e.g., woven), film or plastic. The scrim may provide buoyancy and may aid in flotation of the devices provided herein.

When utilized the scrim may be exposed (i.e., not covered or wrapped in hair) at a first end portion and second end portion. Alternatively, the scrim may be exposed on all ends or edges such that mammalian hair is attached to or wrapped around a middle portion of the scrim. The scrim may include one or more connection means such as ringlets or grommets at each of the first end portion and second end portion. According to one embodiment, the scrim may include at least one connection means at a corner portion of the scrim (e.g., connection means at all four corners).

According to one embodiment, mammalian hair may be punched (e.g., needle punched) into the scrim on a top portion and/or bottom portion of the scrim. According to another embodiment, mammalian hair may be wrapped around or placed on either side of the scrim.

### Methods

A method for containing one or more liquid contaminants in or on one or more aqueous liquids is provided. The method may include the step of deploying a device that includes mammalian hair and at least one scrim, the scrim having a first end portion and a second end portion. The mammalian hair is disposed on or wrapped around the scrim. The scrim includes at least one connection means at each of the first end portion and second end portion. The device is particularly suited to contain one or more liquid contaminants in or on one or more aqueous liquids. According to one embodiment, the device absorbs the one or more liquid contaminants. According to one embodiment, the mammalian hair is utilized as a mammalian hair mat. According to one embodiment, the mammalian hair mat exhibits a hair density of from about 0.02 to about 3 grams of mammalian hair per cubic inch. According to one embodiment, the mammalian hair mat is capable of or otherwise suit for holding and retaining from generally about 0.02 to generally about 15 grams of liquid contaminant per cubic inch of mammalian hair. According to one embodiment, the mammalian hair mat further includes at least one natural fiber from the group including human hair, animal hair, coconut fiber, seaweed, kelp, cotton, corn silk and peat moss. According to one embodiment, the mammalian hair mat may include a plurality of man-made fibers.

Although specific embodiments of the present invention are herein illustrated and described in detail, the invention is not limited thereto. The above detailed descriptions are provided as exemplary of the present invention and should not be construed as constituting any limitation of the invention. Modifications will be apparent to those skilled in the art, and all modifications that do not depart from the spirit of the invention are intended to be included with the scope of the appended claims.

### STATEMENTS OF THE DISCLOSURE

The following statements are provided:

Statement 1. A device is provided for containing or absorbing liquid contaminant in or on an aqueous liquid, the device comprising:
mammalian hair; and
at least one flotation device, the flotation device having a first end portion and a second end portion,
wherein the flotation device include at least one connection means at each of the first end portion and second end portion.

Statement 2. The device of statement 1, wherein the mammalian hair is utilized as the mammalian hair mat.

Statement 3. The device of statement 2, wherein the mammalian hair mat exhibits a hair density of from about 0.02 to about 3 grams of mammalian hair per cubic inch.

Statement 4. The device of statement 2, wherein the mammalian hair mat is capable of holding and retaining from generally about 0.02 to generally about 15 grams of liquid contaminant per cubic inch of mammalian hair.

Statement 5. The device of statement 2, wherein the mammalian hair mat further comprises at least one natural fiber from the group including human hair, animal hair, coconut fiber, seaweed, kelp, cotton, corn silk and peat moss.

Statement 6. The device of statement 2, wherein the mammalian hair mat further comprises a plurality of man-made fibers.

Statement 7. The device of statement 1, wherein the at least one connection means at each of the first end portion and second end portion comprises a ringlet, the ringlet adapted to receive an attachment means.

Statement 8. The device of statement 7, wherein the attachment means includes a rope.

Statement 9. The device of statement 1, wherein the at least one connection means at each of the first end portion and second end portion comprises an eye hook.

Statement 10. The device of statement 1, wherein the liquid contaminant is less dense than the aqueous liquid.

Statement 11. The device of statement 1, wherein the liquid contaminant comprises oil.

Statement 12. The device of statement 1, wherein the flotation device comprises at least one closed-cell foam.

Statement 13. The device of statement 1, wherein the flotation device is mold and mildew resistant.

Statement 14. The device of statement 1, wherein the flotation device comprises polyethylene foam, urethane foam, polyurethane foam, or a combination thereof.

Statement 15. The device of statement 1, wherein the flotation device is substantially cylindrical in shape.

Statement 16. The device of statement 1, wherein the mammalian hair mat is secured to the flotation device.

Statement 17. The device of statement 1, wherein the flotation device is encased within the mammalian hair.

Statement 18. The device of statement 1, wherein the mammalian hair is encased within the flotation device.

Statement 19. A device for containing or absorbing liquid contaminant in or on an aqueous liquid, the device comprising:
mammalian hair; and
at least one scrim, the scrim having a first end portion and a second end portion,
wherein the mammalian hair is disposed on or wrapped around the scrim, and
wherein the scrim includes at least one connection means at each of the first end portion and second end portion.

Statement 20. The device of statement 19, wherein the mammalian hair is utilized as a mammalian hair mat.

Statement 21. The device of statement 20, wherein the mammalian hair mat exhibits a hair density of from about 0.02 to about 3 grams of mammalian hair per cubic inch.

Statement 22. The device of statement 20, wherein the mammalian hair mat is capable of holding and retaining from generally about 0.02 to generally about 15 grams of liquid contaminant per cubic inch of mammalian hair.

Statement 23. The device of statement 20, wherein the mammalian hair mat further comprises at least one natural fiber from the group including human hair, animal hair, coconut fiber, seaweed, kelp, cotton, corn silk and peat moss.

Statement 24. The device of statement 20, wherein the mammalian hair mat further comprises a plurality of man-made fibers.

Statement 25. The device of statement 19, wherein the at least one connection means at each of the first end portion and second end portion comprises a ringlet, the ringlet adapted to receive an attachment means.

Statement 26. The device of statement 25, wherein the attachment means includes a rope.

Statement 27. The device of statement 19, wherein the at least one connection means at each of the first end portion and second end portion comprises an eye hook.

Statement 28. The device of statement 19, wherein the liquid contaminant is less dense than the aqueous liquid.

Statement 29. The device of statement 19, wherein the liquid contaminant comprises oil.

Statement 30. The device of statement 19, wherein the scrim comprises fabric (e.g., woven), film or plastic.

Statement 31. The device of statement 19, wherein the mammalian hair is punched into a top portion and bottom portion of the scrim.

Statement 32. A method for containing one or more liquid contaminants in or on one or more aqueous liquids comprising the step of:
deploying a device comprising:
   mammalian hair; and
   at least one scrim, the scrim having a first end portion and a second end portion,
wherein the mammalian hair is disposed on or wrapped around the scrim,
wherein the scrim includes at least one connection means at each of the first end portion and second end portion, and
wherein the device contains the one or more liquid contaminants in or on the one or more aqueous liquids.

Statement 33. The method of statement 32, wherein the device absorbs the one or more liquid contaminants.

Statement 34. The method of statement 32, wherein the mammalian hair is utilized as a mammalian hair mat.

Statement 35. The method of statement 34, wherein the mammalian hair mat exhibits a hair density of from about 0.02 to about 3 grams of mammalian hair per cubic inch.

Statement 36. The method of statement 34, wherein the mammalian hair mat is capable of holding and retaining from generally about 0.02 to generally about 15 grams of liquid contaminant per cubic inch of mammalian hair.

Statement 37. The method of statement 34, wherein the mammalian hair mat further comprises at least one natural fiber from the group including human hair, animal hair, coconut fiber, seaweed, kelp, cotton, corn silk and peat moss.

Statement 38. The method of statement 34, wherein the mammalian hair mat further comprises a plurality of man-made fibers.

### Example 1

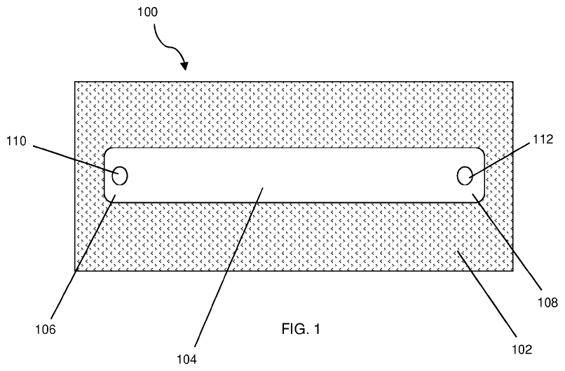

One embodiment of a device 100 for containing and absorbing liquid contaminant in or on an aqueous liquid is illustrated in FIG. 1. The device 100 includes a mammalian hair mat 102 and a flotation device 104 encased within the mammalian hair mat 102. The flotation device 104 includes a first end portion 106 and a second end portion 108. The flotation device 104 includes a connection means (110, 112) at each of the first end portion 106 and second end portion 108. The connection means (110, 112) are illustrated as grommets or ringlets.

### Example 2

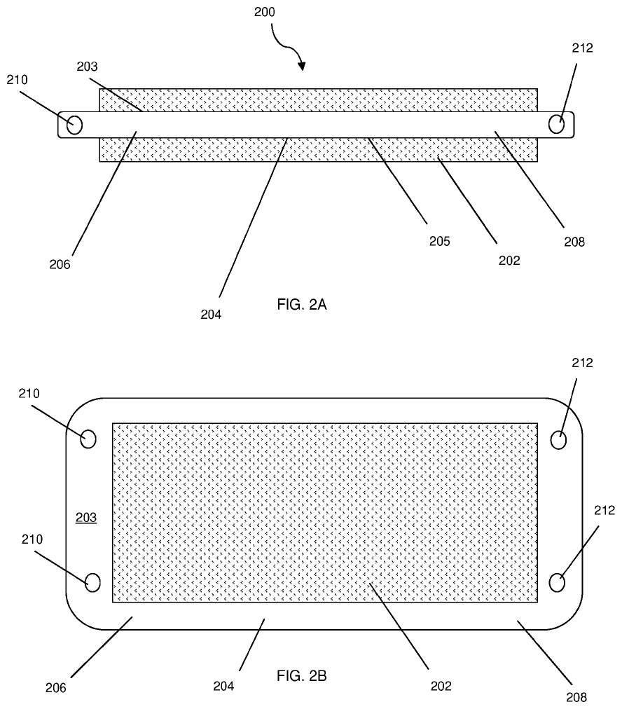

A side view of one embodiment of a device 200 for containing and absorbing liquid contaminant in or on an aqueous liquid is illustrated in FIG. 2A. FIG. 2B provides a top view of the device 200.

The device 200 includes a mammalian hair 202 applied to or wrapped around a scrim 204. The mammalian hair 202 may be a mammalian hair mat that is applied to a top portion 203 and bottom portion 205 of the scrim 204. The mammalian hair 202 may also be punched into the top portion 203 and bottom portion 205 of the scrim 204.

The scrim 204 includes a first end portion 206 and a second end portion 208. The scrim 204 includes one or more connection means (210, 212) at each of the first end portion 206 and second end portion 208. The connection means (210, 212) are illustrated as grommets or ringlets.

### Example 3

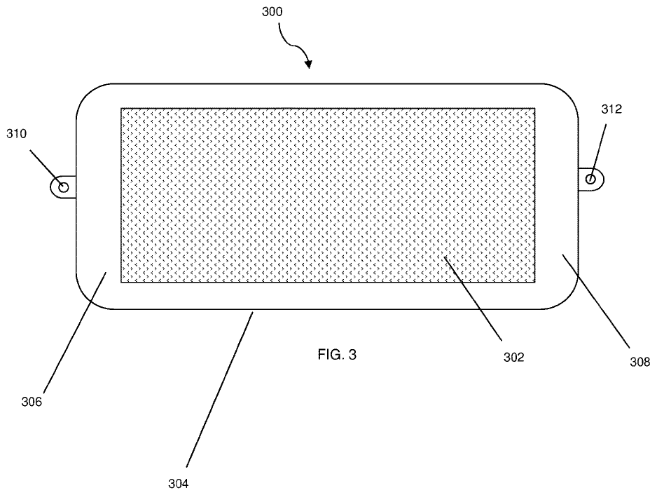

A side view of one embodiment of a device 300 for containing and absorbing liquid contaminant in or on an aqueous liquid is illustrated in FIG. 3. The device 300 includes a mammalian hair 302 disposed within a flotation device 304. The mammalian hair 302 may be a mammalian hair mat.

The flotation device 304 includes a first end portion 306 and a second end portion 308. The flotation device 304 includes a connection means (310, 312) at each of the first end portion 306 and second end portion 308. The connection means (310, 312) are illustrated as grommets or ringlets.

What is claimed is:

1. A device for containing or absorbing liquid contaminant in or on an aqueous liquid, the device comprising:
mammalian hair; and
at least one scrim, the scrim having a first end portion and a second end portion,
wherein the mammalian hair is disposed on or wrapped around the scrim, and
wherein the scrim includes at least one connection means at each of the first end portion and second end portion.

2. The device of claim 1, wherein the mammalian hair is utilized as a mammalian hair mat.

3. The device of claim 2, wherein the mammalian hair mat exhibits a hair density of from about 0.02 to about 3 grams of mammalian hair per cubic inch.

4. The device of claim 2, wherein the mammalian hair mat is capable of holding and retaining from generally about 0.02 to generally about 15 grams of liquid contaminant per cubic inch of mammalian hair.

5. The device of claim 2, wherein the mammalian hair mat further comprises at least one natural fiber from the group including human hair, animal hair, coconut fiber, seaweed, kelp, cotton, corn silk and peat moss.

6. The device of claim 2, wherein the mammalian hair mat further comprises a plurality of man-made fibers.

7. The device of claim 1, wherein the at least one connection means at each of the first end portion and second end portion comprises a ringlet, the ringlet adapted to receive an attachment means.

8. The device of claim 7, wherein the attachment means includes a rope.

9. The device of claim 1, wherein the at least one connection means at each of the first end portion and second end portion comprises an eye hook.

10. The device of claim 1, wherein the liquid contaminant is less dense than the aqueous liquid.

11. The device of claim 1, wherein the liquid contaminant comprises oil.

12. The device of claim 1, wherein the scrim comprises fabric, film or plastic.

13. The device of claim 1, wherein the mammalian hair is punched into a top portion and bottom portion of the scrim.

14. A method for containing one or more liquid contaminants in or on one or more aqueous liquids comprising the step of:
deploying a device comprising:
mammalian hair; and
at least one scrim, the scrim having a first end portion and a second end portion,
wherein the mammalian hair is disposed on or wrapped around the scrim,
wherein the scrim includes at least one connection means at each of the first end portion and second end portion, and
wherein the device contains the one or more liquid contaminants in or on the one or more aqueous liquids.

15. The method of claim 14, wherein the device absorbs the one or more liquid contaminants.

16. The method of claim 14, wherein the mammalian hair is utilized as a mammalian hair mat.

17. The method of claim 16, wherein the mammalian hair mat exhibits a hair density of from about 0.02 to about 3 grams of mammalian hair per cubic inch.

18. The method of claim 16, wherein the mammalian hair mat is capable of holding and retaining from generally about 0.02 to generally about 15 grams of liquid contaminant per cubic inch of mammalian hair.

19. The method of claim 16, wherein the mammalian hair mat further comprises at least one natural fiber from the group including human hair, animal hair, coconut fiber, seaweed, kelp, cotton, corn silk and peat moss.

20. The method of claim 16, wherein the mammalian hair mat further comprises a plurality of man-made fibers.
